(19) United States
(12) Patent Application Publication
(10) Pub. No.: US 2007/0066500 A1
(43) Pub. Date: Mar. 22, 2007

(54) Composition with enhanced squeaky feel

(75) Inventors: Lin Yang, Fort Lee, NJ (US); Kavssery Parameswaran Ananthapadmanabhan, Highland Mills, NY (US); Junqi Ding, Tenafly, NJ (US); Kannapon Lopetcharat, New York, NY (US); Teanoosh Moaddel, Bridgewater, NJ (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: CONOPCO, INC., d/b/a UNILEVER

(21) Appl. No.: 11/231,622

(22) Filed: Sep. 21, 2005

(51) Int. Cl. A61K 8/00

(52) U.S. Cl. 510/130

(57) ABSTRACT

The invention discloses compositions with enhanced squeaky feel, when rinsed in water, defined by a region of a surfactant-cation phase diagram comprising surfactant-cation precipitate and/or surfactant monomer, but substantially no surfactant micelle. The present invention focuses, for example, on the relationship between counter-ion (e.g., cation, preferably salt cation) and surfactant. Specifically, it has been found that enhancing the precipitation of counter-ion-surfactant complex helps reduce surfactant micellar concentration, enhance surface tension and lead to compositions with enhanced “squeaky” feel. The precipitation can in turn be promoted by enhancing surfactant counter-ion interaction, e.g., by increasing sensitivity of surfactant to counter-ion (e.g., by using long chain length hydrophobe group), and/or by preformulating additional counter-ion into the surfactant solution.

### FIELD OF THE INVENTION

The present invention relates to compositions which enhance squeaky feel as well as to processes for enhancing a “squeaky” skin feel desired by consumers but which feel is difficult to obtain in compositions when the surfactant is predominately synthetic surfactant. Specifically, by controlling the interaction between surfactant and cation (e.g., by increasing level of calcium or other cation in the starting surfactant-containing formulation, or by increasing the sensitivity of the surfactant in the formulation to calcium during water rinse), it is possible to have compositions perceived to have enhanced squeaky feel versus slimy feel during rinsing. Specifically, applicants have developed phase diagrams mapping the relationship between surfactant and cation and permitting selection of desired compositions (e.g., having enhanced squeaky feel) when ratios of surfactant to cation are met.

### BACKGROUND

While bars which contain large amounts of predominantly synthetic surfactant are generally milder than soap, one aspect of such bars which many consumers have complained about is that such synthetic bars do not provide the “squeaky”, friction-like feeling (associated with “squeaky” clean) which is associated with soap.

Applicants have now found that the extent of interaction between synthetic surfactant and salt leading to precipitation of surfactant-cation salt (i.e., the sensitivity of the synthetic surfactant to salts, such as calcium salts) directly correlates with the “squeaky” clean perception. While not wishing to be bound by theory, applicants believe this occurs because increasing the concentration of cations decreases the overall amount of surfactant micelle at certain regions of the phase diagram, i.e., at a certain surfactant to cation ratio (the micelle is being consumed in order to form, for example, surfactant-calcium precipitate), thereby increasing surface tension and causing more frictional force. Specifically, in the presence of surfactant micelle, the adsorption of the negatively charged surfactant molecules onto skin surfaces lead to a high repulsion force when the skin surfaces are rubbing against each other; and this high repulsion force often results in the slimy feel experienced by the consumers. By contrast, in the absence of surfactant micelle due to the formation of the surfactant—cation precipitate, both the skin surfaces and the surfactant—cation precipitate become uncharged which results in high friction force when the two surfaces are rubbing against each other and thereby providing the squeaky feel experienced by the consumer.

In short, the higher interaction between surfactant and cations (e.g., calcium) leads to precipitation which can reduce the quantity of surfactant micelles (it is the micelles which are associated with surface activity and continuously charged skin surface) and leads to a “region” where “squeakiness” (apparently through enhanced frictional force) is enhanced.

In view of the theoretical reasons applicants believe to be behind enhanced “squeaky” feeling, applicants have found that promotion of this squeaky sensation can be achieved by enhancing this surfactant-cation interaction, leading to loss of surfactant micelles and early entrance to such squeaky region during rinse/dilution. This enhanced interaction can in turn be promoted, for example, by (1) increasing the sensitivity of the surfactant to cations, such as calcium (hastening the formation of surfactant-calcium precipitates and loss of surfactant micelles) and/or by (2) preformulating, for example, calcium salt into a surfactant formulation used in the composition (again hastening loss of surfactant micelle as surfactant monomers break away from the micelle to form surfactant-calcium precipitate).

Yet another way to reduce or eliminate surfactant micellar structure is to use surfactants of low Krafft Temperature (the temperature above which surfactant crystals are dissolved to form a micellar solution). When surfactants are more readily dissolved, e.g., at lower Krafft temperature, absence of surfactant crystal structures, which may serve as a reservoir of micelles during rinse, leads to more “squeaky” feel. Surfactant micellar solid structure can also be lost or broken up (leading to less sliminess and more squeakiness) using techniques such as surfactant blending, use of cosolvents or use of small molecular additives.

Applicants are aware of no art which recognizes the relationship between surfactant and cation interaction (leading to formation of surfactant-cation precipitation at the expense of micelles) in enhancing “squeaky” feel and which discloses a process to enhance such interaction.

WO 2002/12430 (Unilever) discloses synthetic bar compositions comprising anionic surfactant, soap, free fatty acid and a divalent cation source such as calcium salt. There is no recognition of a specific region where surfactant micelles are no longer present and squeakiness is enhanced, or of a process for enhancing squeaky feel by hastening entrance into this substantially micelle-free region.

Other references are noted as follows:

JP 05271697 (Kao) discloses soap composition containing soap of sodium, potassium, and magnesium and/or calcium oxide, foaming well and not cracking.

Patent GB 2253404 (Kao) discloses detergent bar compositions containing magnesium oxide and/or calcium oxide, which maintain bar shape during use, without swelling, liquefaction or cracking.

WO 98/38269 (Procter & Gamble Company) discloses a laundry detergent bar with a calcium salt and siliceous material complex formed in situ.

Somasundaran, P. Ananthapadmanabhan, K. P., Celik, M. S., “Precipitation-Redissolution Phenomema in Sulfonate-AlCl₃ Solution” Langmuir, 1988, 4, 1061-1063.

Noik, C., Baviere, M., Defives, D., “Anionic Surfactant Precipitation in Hard Water” Journal of Colloid and Interface Science, 1987, 115, 35-45.

Chou, S. I. Bae, J. H., “Surfactant Precipitation and Redissolution in Brine” Journal of Colloid and Interface Science, 1983, 96, 192-203.

Fujiware, M. Miyake, M. Abe, Y., “Colloidal Properties of V-sulfonated Fatty Acid Methyl Esters and Their Applicability in Hard Water” Colloid & Polymer Science, 1993, 271, 780-785.

Peacock, J. M. Matijevic, E., “Precipitation of Alkylbenzene Sulfonates with Metallons” Journal of Colloid and Interface Science, 1980, 77, 548-554.

Two co-pending applications by applicants which mention squeakiness (obtained with different compositions/mechanisms) are U.S. Ser. No. 10/883,326 to Morikis et al., entitled “Mild Synthetic Detergent Toilet Bar Composition”; and U.S. Ser. No. 11/075,226 to Moaddel et al., entitled “Mild, Low Soluble Soap Bars Which Have Non-Slimy Quick Rinse Perception in Use”.

In none of the references noted is there disclosed the relationship between squeaky feel and diminution (e.g., substantial elimination) of surfactant micellar concentration. There is also not disclosed a process or method of controlling squeaky feel by (a) enhancing the sensitivity of surfactant to cation, such as calcium (causing calcium-surfactant complex which dominates or swamps out the quantity of micelle); or (b) by enhancing cation concentration in the surfactant. Further there is not disclosed phase diagrams which map out ratios of surfactant to cation so that one can select formulations with desired skin feel attributes merely by choosing formulations with ratio of surfactant to cation set forth in the phase diagram.

### BRIEF DESCRIPTION OF THE INVENTION

The subject invention relates to cleanser compositions comprising at least one anionic surfactant and a sufficient amount multivalent cation containing salt such that the cleanser composition, during rinsing, passes through a region of the phase diagram where precipitation of surfactant-multivalent occurs and the solution is substantially depleted of micelles, said depletion occurring at a dilution factor less than would be required to obtain the same substantially micelle-free solution if the multivalent cation containing salt were not present.

In a second embodiment, the subject invention relates to a process for enhancing “squeaky” feel (measured by acoustic means or by panel testing) by selecting a ratio of surfactant to cation which will place the composition in a region which is “squeaky” as predicted from a phase diagram. Generally, it is predominantly synthetic surfactants (surfactant system comprising >50% synthetic and <50% soap) which obtain greater “squeakiness” because compositions where surfactant system is predominantly soap (e.g., greater than 70%, preferably greater than 75%, more preferably >80%) are already in the desired “squeaky” region under normal water hardness condition (e.g., about 30 to 150 ppm calcium). However, even at levels as low as 20% surfactant and 80% soap, some effect should be observable since increasing the squeakiness of any amount of slimy compound, no matter how small, has some effect. The squeaky feeling is desired by many consumers and is viewed as a cue for good cleansing.

Specifically, by identifying the relationship (ratios) between surfactant and cation salt, (e.g. calcium or aluminum salts), applicants have found that controlling the surfactant-cation interaction (e.g., by increasing the surfactant sensitivity to cation or by increasing the quantity of cation in the surfactant solution) leads to enhancing squeaky sensation. As indicated, this is believed to occur because of substantial elimination of surfactant micelle which micelles, in turn, are responsible for slimy feel.

These and other aspects, features and advantages will become apparent to those of ordinary skill in the art from a reading of the following detailed description and the appended claims. For the avoidance of doubt, any feature of one aspect of the present invention may be utilized in any other aspect of the invention. It is noted that the examples given in the description below are intended to clarify the invention and are not intended to limit the invention to those examples per se. Other than in the experimental examples, or where otherwise indicated, all numbers expressing quantities of ingredients or reaction conditions used herein are to be understood as modified in all instances by the term “about”. Similarly, all percentages are weight/weight percentages of the total composition unless otherwise indicated. Numerical ranges expressed in the format “from x to y” are understood to include x and y. When for a specific feature multiple preferred ranges are described in the format “from x to y”, it is understood that all ranges combining the different endpoints are also contemplated. Where the term “comprising” is used in the specification or claims, it is not intended to exclude any terms, steps or features not specifically recited. All temperatures are in degrees Celsius (° C.) unless specified otherwise. All measurements are in Si units unless specified otherwise. All documents cited are—in relevant part—incorporated herein by reference.

### BRIEF DESCRIPTION OF DRAWINGS

FIG. 1 is a schematic figure showing how, as micelle is disappearing (surfactant concentration decreasing), the continuous charges formed between the skin surfaces and particles disappear (both become uncharged) thus tending to cause friction/squeakiness.

FIG. 2(a) is a surfactant salt phase diagram showing 4 regions (e.g. region A is single-phase region where surfactant micelles and monomers exist) as surfactant concentration decreases. Formulation 1 is a surfactant composition preformulated with calcium salt. Formulation 2 is a surfactant composition with little salt. As seen, Formulation 1 enters through a squeaky region (gray region C) with much less dilution than to Formulation 2. The fewer dilutions and relation to surface tension is also clearly seen in FIG. 2(b).

FIG. 3 shows the squeakiness boundary (empty dots and dashed line) and the equilibrium precipitation boundary (black dots and solid line) of an anionic surfactant, sodium dodecyl sulphate (SDS) and CaCl₂ solution at 25° C. As seen, the squeakiness boundary is much narrower than the precipitation boundary in the sense that the squeakiness boundary covers a much smaller area than the precipitation boundary in the phase diagram.

FIG. 4 shows the squeakiness boundaries of Jordapon, DEFI and SDS at room temperature and shows how the squeakiness boundary may depend on the surfactant used (e.g., SDS versus DEFI versus Jordapon). From FIG. 4, it can be seen that, if we have a 0.75% surfactant solution, around 0.12% CaCl₂ is needed to precipitate SDS, 0.2% for DEFI (less calcium sensitive) and 0.25% for Jordapon. So for the same surfactant concentration, more Ca²⁺ is needed to precipitate DEFI than SDS, and even more for Jordapon.

DEFI: Directly Esterified Fatty Isethionate, usually have around 75% of SCI (sodium cocoyl isethionate) and the rest fatty acid and other impurities; Jordapon: a brand name of the SCI containing chemical purchased from supplier. Usually have 87% SCI and the rest fatty acid and other impurities.

FIG. 5 is the acoustic profiles in different regions of SDS—calcium chloride phase diagram following dilution route 1. I represents the sound profile which corresponds to the region A in FIG. 2. II represents the sound profile which corresponds to region B referred to in FIG. 2. III corresponds to region C at a point close to the B/C boundary in FIG. 2. IV corresponds to a point in region C close to the C/D boundary. V corresponds to a point in region D in FIG. 2. VI is the sound profile of Ca-water solvent. As shown by this set of data, Formulations in region C of the phase diagram, which is defined as the squeaky region per the surface tension analysis, are indeed squeaky as indicated by a higher sound pressure (Pa).

FIG. 6 is the acoustic profiles in different regions of SDS—calcium chloride phase diagram following dilution route 2. I to III are the SDS/water solutions (the concentrations are above critical micelle concentration, or CMC); IV corresponds to a point where the solution is close to CMC. V corresponds to a point where the solution concentration is below CMC. VI is the pure water solvent. The data show that, without calcium chloride preformulated into the surfactant solution, squeakiness is not achieved until the CMC of the surfactant is reached (which means more dilutions are required).

FIG. 7 is the acoustic profiles of DEFI (dilution route 2, FIG. 7A) vs. DEFI with fatty acid and calcium chloride preformulated (dilution route 1, FIG. 7B)). The figures demonstrate that for dilution route 1, less than 10 seconds are needed to achieve squeaky feel, as indicated by a stronger sound pressure (e.g., Pa reaching around 100); while for dilution route 2, squeaky feel is not achieved until the end of the experiment (25 seconds). Replacing calcium with an equivalent amount of triglyceride oil illustrates the importance of calcium in inducing precipitation of SCI leading to squeaky-clean perception.

FIG. 8 is the panel results of the comparison of the relative squeaky-clean elicited by a cleaning bar containing 50% DEFI (sodium cocoyl isethionate) and another bar of same composition, but preformulated with 4% CaCl₂. Results show that the bar preformulated with 4% CaCl₂ is perceived as squeakier (as seen by higher relative % of people who perceived it as squeaky, using pure soap bar, as defined in examples, as a control).

Formulation of DEFI Bar (without 4% CaCl₂)

| Ingredient | % |
|---|---|
| Sodium cocoyl isethionate | ≈50% |
| Free fatty acid | ≈23% |
| Fatty acid soap | ≈7% |
| Sodium isethionate | ≈5% |
| Sodium stearate | ≈3% |
| Betaine | ≈3% |
| Water & minors | Balance |

(when CaCl₂ is used, all other ingredients are lowered proportionally)

Formulation for Soap Bar (control against DEFI bars)

| Ingredient | % |
|---|---|
| Fatty acid soap | ≈77% |
| Free fatty acid | ≈8% |
| Water & minors | Balance |

FIG. 9 shows how cation sensitivity and Krafft temperature vary depending on chain length of surfactant. Thus, generally, higher chain length is more sensitive and will form precipitate (resulting in squeaky feel) while lower chain length is less sensitive (resulting in more micelle & slimier feel). On the other hand, there are fewer molecules available using the lower chain length molecules (they have lower Krafft temperature and most micelles will be rinsed away) and fewer micelles is associated with squeakier feel. Thus, these two factors balance each other.

FIG. 10 shows how sliminess (or squeakiness) varies based on chain length. It factors in competing effects of chain length alone and Krafft temperature.

FIG. 11 shows the calcium tolerance of various surfactants. Generally, the greater the tolerance (less precipitate formed), the “slimier” the feel.

FIG. 12 shows the dependency of calcium tolerance on fatty acid content of surfactant. Fatty acid tends to reduce cation tolerance, cause micellar break-up (e.g., fatty acid help precipitate form) and enhance squeaky feel.

FIG. 13 shows surface tension of SDS (sodium dodecyl sulphate)—CaCl₂ solution (at constant CaCl₂ concentration of 0.1 wt. % CaCl₂) as function of SDS concentration.

FIG. 14 is a schematic of the set-up used to follow product evolution on dilution for rinse off product. One arm with cleansing product is immersed in a tank filled with water of a given hardness and temperature. A hydrophone is also immersed in the water. Accelerometers were attached to the arm skin surface. The other hand rubs the arm with product while both the “rubbing” sound is picked up by the hydrophone and skin vibration by the accelerometers simultaneously.

FIG. 15 is schematic of finger-tip acoustic measurement.

FIG. 16 discloses properties of SDS-calcium precipitate at constant calcium chloride concentration (0.1 wt. % CaCl₂) as a function of SDS concentration; the amount of precipitation; and mobility of the precipitate.

FIG. 17 is results of perceived squeaky feel by Japanese Panel in three different regions.

### DETAILED DESCRIPTION OF THE INVENTION

In one embodiment, the invention relates to compositions which, if they exist within a defined phase diagram area, have enhanced squeakiness relative to compositions outside the defined phase diagram area. The phase diagram area of enhanced “squeakiness” defines a region comprising surfactant-cation precipitate and/or surfactant monomer but substantially no surfactant micelle. The substantial absence of micelles (e.g., either because surfactant is only in form of monomers co-existing with dissolved cation in clear solution; or that surfactant is present in the form of surfactant-cation salt precipitate co-existing with surfactant monomers in cloudy solution) which defines the “squeaky” region (non-micellar region). Thus, since each surfactant may have different sensitivity to ion precipitation, it is not possible to precisely define exactly how much cation or exactly how much surfactant is needed to ensure a solution substantially free of all micelles. As indicated, however, in the substantial absence of these micelles, squeaky behavior is exhibited.

Further, the phase diagram defines a region where this precipitation occurs and there is substantial depletion of micelles, said depletion occurring at a dilution factor less than would be required to obtain the same substantially micelle-free solution if a sufficient amount of the multivalent cation containing salt were not present.

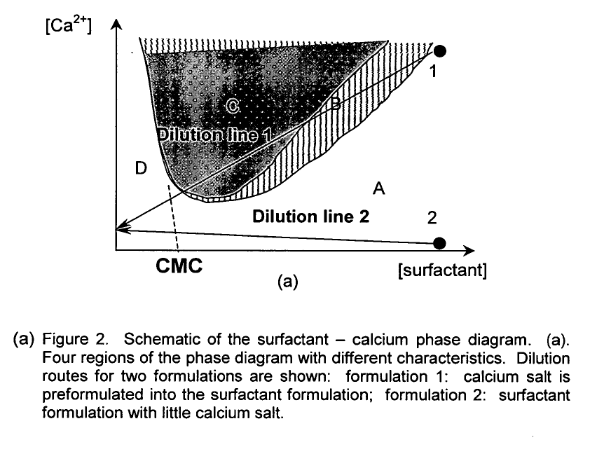

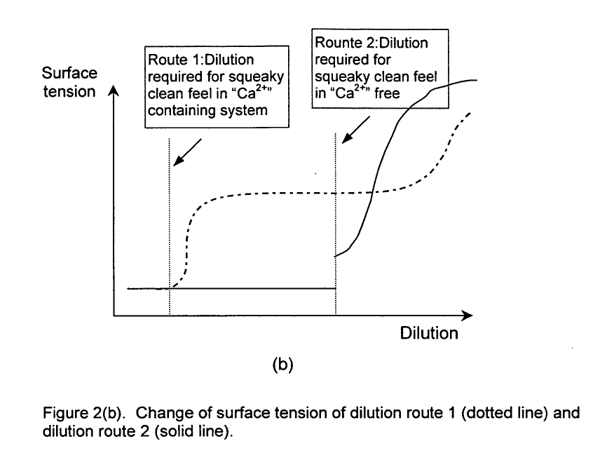

This above concept may perhaps be best exemplified by referring to FIG. 2 relating to regions A, B, C and D in connection with the Table set forth below.

| Non-squeaky/slimy feel (micellar) | | Squeaky clean feel (non-micellar) | |
|---|---|---|---|
| Region A | Region B | Region C | Region D |
| Surfactant | Surfactant | Surfactant | Surfactant |
| micelles and | micelles and | monomers co- | monomers and |
| monomers co- | monomers co- | exist with | dissolved |
| exist with | exist with | surfactant - | Ca²⁺ ions |
| dissolved | surfactant | calcium salt | coexist. |
| Ca²⁺ ions | calcium salt | precipitate | |
| | precipitate | | |
| Clear solution | Cloudy solution | Cloudy solution | Clear solution |

In a second embodiment, the present invention relates to a process for enhancing “squeaky” feel of a cleansing system comprising predominantly synthetic surfactant (as indicated earlier, the effect should be observable no matter how little surfactant is present but, as a practical matter, synthetic is greater than 20% of synthetic/soap system, preferably greater than 50%, because the soap present already is cation sensitive and will exhibit squeaky behavior).

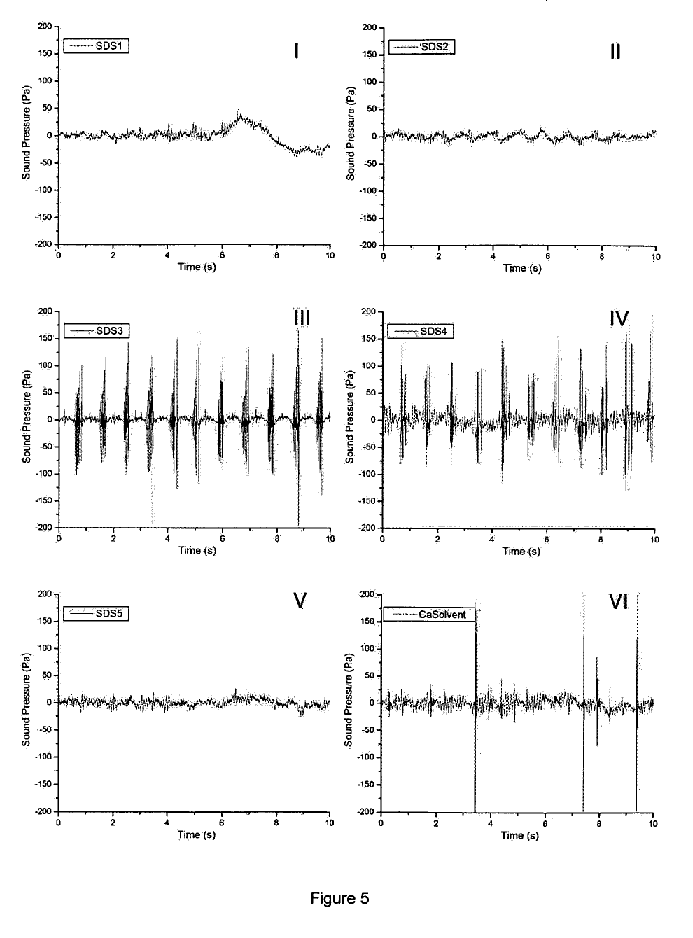

More specifically, by enhancing the sensitivity (e.g., by using longer chain length groups which precipitate more readily; by increasing amount of fatty acid in surfactant solution) of surfactant to cation, (e.g. calcium, aluminum, magnesium, zinc) and/or by enhancing the amount of cation in the surfactant containing formulation used (e.g., liquid or solid), applicants have found it is possible to enhance the “squeakiness” (function of friction against skin) of the composition. This can be seen for example, from FIGS. 5 and 2 discussed above and herein. From a comparison of FIG. 5 and FIG. 2, it can be seen that, where there is substantially no micelle present (regions C & D in FIG. 2), the greatest acoustic “squeakiness” is seen (see region III and IV in FIG. 5).

More specifically, based on various studies/experiments, applicants generally determined that, in the presence of ionic salt (e.g., cation such as calcium or poly-cation), the properties (e.g., squeakiness) of a surfactant molecule absorbed onto a skin surface may be altered. Specifically, the surface tension (which reflects the surface activity of the surfactant) of a solution containing surfactant salt mixture (e.g., surfactant-calcium salt) was studied to understand how the interaction of surfactant and cation (e.g., calcium ions) affected both the rinsability and perceived properties of the surfactant.

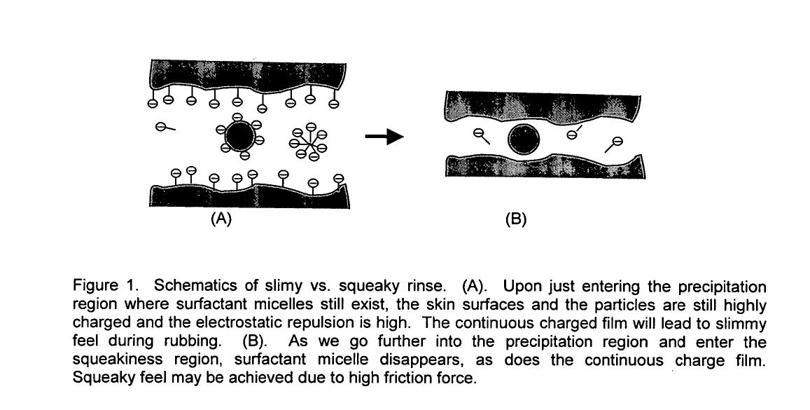

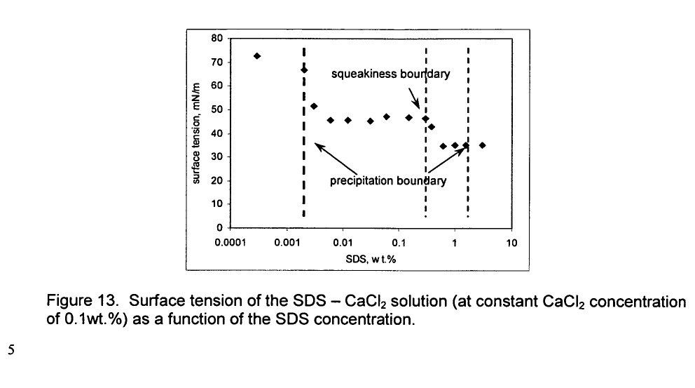

Applicants unexpectedly found that there is a squeakiness region where the surfactant is “consumed” by ions as a surfactant-cation precipitate begins to form. As already indicated, this region is defined by the substantial absence of surfactant micelles even if there is some surfactant monomer present. Applicants have found that in this region, the surface activity of the surfactant becomes low and this leads to “squeakiness”. Without wishing to be bound by theory, applicants believe that the squeakiness occurs by one or both of the following mechanisms:
(1) when surfactant micelles are present (and normally when surface tension is low), surfactant which is adsorbed onto the skin surfaces (e.g., when rubbed by the consumers) forms a continuously charged film, the repulsion force between which causes slimy sensory feel; when the surfactant micelle starts to disperse (in presence of cation) by forming surfactant-cation precipitate, the film is broken, and surface tension increases; this dewetting (e.g. increase in surface tension) causes more frictional force which is perceived as “squeaky” (FIG. 1) (see also FIG. 2 and FIG. 13 showing schematic and measured data of changing surface tension in presence of cation); and
(2) the uncharged surfactant-cation precipitate (again formed in presence of cation) can further increase the frictional force during rubbing.

More generally, applicants have found that, based on the physiochemical characterization of a surfactant salt phase diagram (e.g., surfactant-calcium phase diagram), a surfactant phase diagram can be divided into 4 regions which are seen schematically in FIG. 2. These regions can be further defined as follows:

Region A (clear solution region): at high surfactant concentration side, the surfactant—cation mixture is a clear solution where surfactant molecules exist as micelles and monomers, and cations (e.g., calcium) behave as counterion.

Region B (precipitation region): as the surfactant concentration decreases, calcium-surfactant salt precipitates as a separate phase in equilibrium with surfactant micelles and monomers.

Region C (squeakiness region): as the surfactant concentration continues to decrease, the insoluble cation-surfactant salt is formed primarily at the expense of surfactant micelles. At certain surfactant concentration, the micelles get consumed completely, leaving monomer in equilibrium with the cation-surfactant salt precipitation. This is the on-set of the squeakiness region.

Region D (singe-phase region): at extremely low surfactant concentration where the surfactant concentration is below what is required by the solubility product to form cation—surfactant salt, single phase region exists, where cations coexist with surfactant monomer.

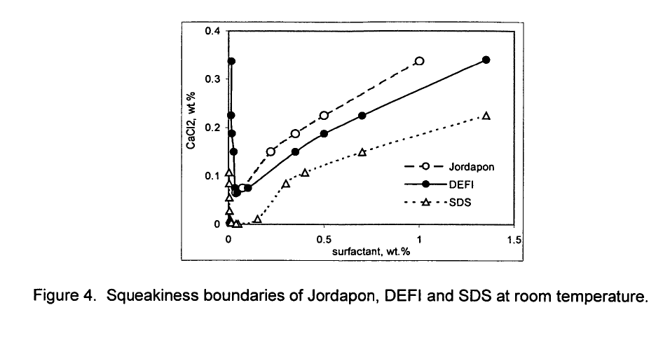

This link between the surfactant-cation (e.g., calcium, aluminum etc.) phase diagram and sensory feel is previously unknown. As part of this invention, applicants have constructed surfactant-cation phase diagrams by experimental means and analyzed different regions of the diagram. In doing so, applicants have found that compositions found within certain regions have superior squeakiness characteristics. For example, FIG. 4 shows squeakiness boundaries of sodium dodecyl sulfate versus two types of sodium acyl isethionate, one having more fatty acid impurities than the other. The figure shows that, compared to SDS, squeakiness boundaries of SCI type (sodium cocoyl isethionate) surfactant covers a much smaller area in phase diagram (e.g., for 0.75% surfactant solution, around 0.12% CaCl₂ needed to precipitate SDS; 0.2% for DEFI and 0.25% for Jordapon).

At a certain surfactant concentration, surface tension doesn't drop until a relatively high calcium salt concentration is reached. In other words, the SCI surfactants are less calcium sensitive (won't form precipitate as readily) compared to SDS surfactant. That probably is one of the reasons why SCI types of surfactants are perceived as slimy during rinsing under normal water hardness. Also, it is noticed that compared with Jordapon, the squeakiness region of DEFI (which has higher fatty acid content) shifts to slightly higher surfactant concentration under certain calcium concentration. It is well known that SCI forms a solid complex structure with fatty acid, which might increase its calcium sensitivity to certain extent.

The subject invention is directed both to compositions as well as to a process to achieve squeaky rinse feel through a better understanding of the surfactant—cation interaction noted above (using, for example, sodium dodecyl sulphate, SDS, as surfactant and calcium chloride as salt). As illustrated in the schematic surfactant—calcium phase diagram (FIG. 2), dilution of a surfactant formulation containing a cationic salt (e.g., calcium salt) during rinsing (point 1 in FIG. 2(a)) will allow for a rapid entry into the precipitation region (region B in FIG. 2(a) followed by the squeakiness region (region C in FIG. 2(a)). Therefore, much less dilution is needed for surfactant containing formulation such as at Point 1 in FIG. 2(a) to reach the high surface tension (as shown in FIG. 2(b)) and a squeaky/clean rinse can be perceived much quicker during rinsing.

On the other hand, for a formulation with little or no salt in the surfactant containing formulations (such as point 2 in FIG. 2(a)), it takes far more dilution to go into the region (region “D” in FIG. 2(a)) where the surfactant micelle disappears to reach a high surface tension (FIG. 2(b)) and perceive squeaky feel during rinsing. Therefore, one way of achieving squeaky feel during rinsing is to preformulate cationic salt (e.g. calcium) into the surfactant containing formulation.

Applicants also did a study of phase diagram using commercially available surfactant, DEFI and Jordapon, which have sodium cocoyl isethionate (SCI) as the major surfactant (75% and 89% respectively) rather than sodium dodecyl sulfate (SDS).

As noted earlier, from FIG. 4 it was found that, compared to SDS, the squeakiness boundary of SCI type of surfactants cover a much smaller area in phase diagram (i.e., smaller area where squeakiness is found). At a given surfactant concentration, surface tension doesn't drop until a relatively high calcium salt concentration is reached. It should be noted that boundaries were drawn by measuring the surface tension in the phase diagram space (see, for example, FIG. 13).

In other words, SCI type of surfactants are less calcium sensitive (more difficult to form calcium/surfactant precipitate) compared to SDS. While not wishing to be bound by theory, applicants believe that this probably is one of the reasons why SCI types of surfactants are perceived as slimy rather than squeaky during rinsing under normal water hardness. Applicants also found that, compared with Jordapon™ (one supplier of sodium cocoyl isethionate), DEFI (which has a higher fatty acid content compared to Jordapon) has the squeakiness region shift to a slightly higher surfactant concentration under given calcium concentration. It is well known that SCI forms a solid complex structure with fatty acid, which might reduce its apparent surfactant activity and increase its calcium sensitivity to certain extent. Thus, the isethionate with more fatty acid will precipitate more easily and be perceived as more “squeaky”.

To demonstrate the different regions defined by surfactant—salt phase diagram, applicants conducted quantitative measure of squeakiness using Acoustic technique in regions illustrated by the surfactant—Calcium phase diagram, using SDA-calcium phase diagram as an example. Applicants also performed Acoustic test during a fore-arm wash test using DEFI formulations with and without calcium salt to demonstrate the difference in squeakiness for routes 1 and 2 illustrated in the phase diagram.

To further support the findings, applicants conducted consumer testing using a trained Japanese panel to score the squeakiness of the regions defined by the surfactant—calcium phase diagram and the difference between a DOVE bar with calcium salt preformulated vs. a DOVE bar without calcium salt.

Besides the surface activity of the surfactant (which applicants linked to sensory feel through an understanding of the surfactant-salt phase diagram), applicants also found that how easily surfactant crystals deposit (larger surfactant crystals deposit more readily) onto the skin surface may also drive the perception of squeaky feel versus slimy feel during the rinsing process. The deposited surfactant crystal is closely related to the structural nature of the surfactant containing formulation. Thus, for example, industrial grade SCI surfactant has a Krafft temperature (which is the temperature above which surfactant crystals readily disperse into solution) above room temperature which means, at room temperature, SCI forms crystals in the solution that may have a high potential to deposit onto skin during wash and rinsing (it is believed high Krafft temperature results in existence of more crystals—i.e., more crystal at room temperature—leading to more deposition, more difficulty to rinse and, therefore, more slime; however, even at high K.T., if sensitivity to cation is high, e.g., soap, the overall result can be squeaky). With a higher percent of deposition (due to high K.T.) and a low calcium sensitivity, this would, therefore, be more likely perceived as “slimy”.

In more general terms, if a relatively large amount of crystals are deposited onto skin (again due to high Krafft temperature or K.T.) and these crystals represent surface-active materials (such as surfactant), upon dilution with water during rinse, surfactant is released continuously into the water solution, and this maintains a relatively high surfactant concentration locally. The electrostatic repulsion between skin surfaces will be high due to the charged crystal deposition and the absorbed surfactant double layer and slimy feel will be perceived (i.e., higher KT equals more surfactant crystals equals “slimier” feel).

However, the surfactant calcium sensitivity (as noted above with regard to soap), of course, also plays an important role in affecting the properties of the surfactant surface activity and that of the deposited surfactant crystal film. If the surfactant is extremely calcium sensitive, the surfactant activity in water solution will be low (fewer micelles, more precipitate), and the surfactant crystals will also be predominantly covered by the uncharged surfactant—calcium salt. Thus repulsion force between two skin surfaces will be low, which can also lead to squeaky feel during rinsing, even though there may have been a high Krafft temperature. Therefore, in terms of sensory feel during wash, both surfactant calcium sensitivity and the structure formed by the surfactant (how much surfactant crystal is present based on Krafft temperature) are two intrinsically related aspects.

Among surfactants (e.g., sodium cocoyl isethionate, or SCI surfactants), different chain lengths and/or structure of the surfactants also affects surface tension and thus rinsing. For example, when small chain length SCI (C₁₀ and below) is used, even though cation (e.g., calcium) sensitivity is low (leading to non-squeaky or “slimy” perception because there are more micelles and less cationic surfactant precipitate), squeakiness is in fact delivered because there is little or no crystal surfactant structure at room temperature (i.e., the lower Kraft temperature of shorter chain length means crystals are dissolved readily at lower temperature). On the other hand, high chain length surfactant (C₁₆ and above) has high K.T. and crystal structure (normally associated with “slimy” because of presence of surfactant crystals), but here squeakiness is driven by the fact that this surfactant is cation sensitive (high chain length more likely to form precipitate complex and fewer micelles). The least squeaky surfactants are at intermediate chain length (e.g., C₁₂ and C₁₄) where neither cation (e.g., calcium) sensitivity (not large enough to form precipitate associated with “squeakiness”) nor crystal structure (Krafft temperature not low enough to have absence of surfactant crystal structure associated with “squeakiness”) is driving squeakiness.

In short, the overall learning was that squeaky feel of surfactant solution can be improved (1) by promoting surfactant-cation interaction (e.g., by increasing cation sensitivity, for example, by increasing chain length of surfactant, e.g., from C₁₂ to C₁₆, or preformulating cation into surfactant formulation) or (2) by breaking the surfactant solid structure to reduce deposition of surfactant solid onto skin, for example, by using small chain length molecules having low K.T., e.g., C₁₀ or below; or by using surfactant blending, or using cosolvent or small molecular additives. This is summarized below:

(1) Enhancing surfactant-cation interaction:
(a) increase cation sensitivity (e.g., for SCI type of surfactant, increase from C₁₂ to C₁₆, but not from short, which is already sensitive, to C₁₂ or C₁₄);
(b) preformulate cation in surfactant solution;
(c) using a salt that leads to higher sensitivity toward the surfactant;

(2) breaking surfactant solid structure;
(a) using small chain length and low Kraft Temperature (K.T.);
(b) using surfactant blending;
(c) using cosolvent;
(d) using small molecular additives.

In general, the higher the cation tolerance (e.g., calcium insensitive), the more difficult it will be to form a precipitate, and the more likely surfactant micelles are to remain intact; this leads to less surface tension, and it is less likely the surfactant will be perceived as squeaky (rather it will be perceived as “slimy”). Conversely, with low cation tolerance (calcium sensitive), the surfactant micelle will tend to dissipate and form precipitate which tend to be perceived as “squeaky”. This perception is further affected by whether the surface active surfactant crystal will deposit onto skin which in turn is a function of K.T. (lower K.T. equals fewer crystals and less deposition and thus it is “squeaky” perceived).

In general, compositions of the invention are defined, as noted above, by those falling within a region of the surfactant-cation phase diagram which is a region (e.g., two-phase region) comprising surfactant-cation precipitate and surfactant monomer, but substantially no surfactant micelle.

The squeaky region can define a precipitate complex formed by the interaction of anionic surfactant salt and thus the increase of surface tension.

Anionic surfactant can be aliphatic sulfonates (e.g., primary alkyl sulfonates or disulfonates, alkyl glyceryl ether sulfonates), or aromatic sulfonates such as alkyl benzene sulfonates.

It may be alkyl sulfate (e.g. C₁₂-C₁₈ alkyl sulfate) or alkyl glyceryl ether sulfates.

Further, it may be alkyl sulfosuccinate; alkyl and acyl taurates, alkyl and acyl sarcosinates, sulfoacetates, alkyl phosphates; phosphate esters, lactates, succinates, maleates, sulfoacetates, alkyl glucosides, acyl isethionates or any of the thousands of anionics such as are well known and well understood by those skilled in the art.

The counter-ion can be any ion which will cause the surfactant to precipitate into the region of the phase diagram where, as noted, there is substantially no micelle.

Examples of counter-ion for anionics include salts such as calcium, aluminum magnesium and zinc salt.

Except in the operating and comparative examples, or where otherwise explicitly indicated, all numbers in this description indicating amounts or ratios of materials or conditions or reaction, physical properties of materials and/or use are to be understood as modified by the word “about”.

Where used in the specification, the term “comprising” is intended to include the presence of stated features, integers, steps, components, but not to preclude the presence or addition of one or more features, integers, steps, components or groups thereof.

The following examples are intended to further illustrate the invention and are not intended to limit the invention in any way.

Unless indicated otherwise, all percentages are intended to be percentages by weight. Further, all ranges are to be understood to encompass both the ends of the ranges plus all numbers subsumed within the ranges.

### EXAMPLES AND PROTOCOL

Materials

SDS=dodecyl sulfate sodium salt, >99%
CaCl₂=Calcium chloride
AlCl₃=Aluminum chloride
SCI=sodium cocoyl isethionate
ASAD=mixture of fatty acids
82/18 soap=tallow soap
AIT=sodium isethionate salt
DEFI=directly esterified isethionate ester, usually with 75% of surfactant and the rest is fatty acid and other inpurity
Jordapon=Brand name of SCI purchased; usually about 87% SCI and rest fatty acid and other impurities
CAS=Cocamido sulfosuccinate
CMC=Critical micelle concentration
   Turbidity Test: Definition of Precipitation Region:

Various points on the phase diagram were obtained by mixing solution of surfactant with the desired concentration of calcium chloride. These were further diluted with solution of the same calcium chloride concentration to arrive at different points at a constant calcium level. The change in turbidity was observed visually (at high surfactant concentration side) or by light scattering (at low surfactant concentration side). Precipitation boundaries of the precipitation region (surfactant solution+surfactant/calcium precipitate) were constructed based on those samples which are turbid.

Surface Tension Test: Definition of Squeakiness Region:

The surface tension was measured by the drop weight method using a Gilmont 0.2 ml micrometer syringe at room temperature. A series of formulations with the same calcium concentration but different surfactant concentrations were measured for surface tension. All samples were filtered through a 0.45 syringe membrane filter once. As the surfactant concentration is lowered from high values at a plateau concentration, surface tension begins to increase. This happens at some surfactant concentration below the onset of surfactant-Ca precipitation. The surfactant concentration of that certain calcium concentration that the surface tension reaches the plateau value was taken as the boundary of the squeakiness region. The above series were then repeated different CaCl₂ levels.

Protocol for Surfactant Squeakiness Test

Eight subjects were recruited from a lab. Their forearms were washed with a soap bar and they were asked to remember the squeaky feel from a soap bar. The clinician dosed at room temperature 2 ml 5% SCI type of surfactant (sodium alkyl isethionate of different chain length) solution (at room temperature) onto the wetted forearm and rubbed into lather. The panellist was asked to start rinse under tap water and start the timer at the same time. The panellist called for stop when he/she felt it was squeaky enough to stop rinse. The time needed for SCI type of surfactants to be rinsed is thus obtained and used as a standard rinsing time. The same forearm was washed with a soap bar again and completely rinsed. Above steps were repeated with another surfactant solution and the time needed to stop rinse was recorded. Each forearm was used twice a day. The slimy score was calculated as the time needed to stop rinse for a surfactant solution divided by that for a SCI solution. Therefore, the sliminess score for SCI is one. The higher the slimy score, the longer it took to rinse the surfactant off and get the squeaky feel.

Surfactant Tolerance for Metal Ions

3 wt. % of different surfactant water solutions were made at room temperature. For those forming a cloudy solution, the upper layer of the surfactant solution was filtered through a 0.45 syringe membrane filter. Calcium chloride solution was titrated into the filtered surfactant solution until the solution turned cloudy. The calcium ion tolerance was then calculated as gram of calcium ion per gram of surfactant.

Acoustic Measurement

Sensory acoustics is a sensitive method for following consumer perception of rinse during the use of wash-off or leave-on products. The method detects an acoustic signal during touch to assess the in-use sensory performance of personal care products and allows one to extract specific sentry attributes or, more specifically, a sensory profile. Acoustic probes (e.g., hydrophone, microphone and/or accelerometer) were placed near the site where two skin surfaces rub against each other to detect the noise or vibration generated by the rubbing. The signal was amplified and conditioned to an Analog-to-Digital board and converted to a digital signal. The digital signal was stored and analyzed by home-made software. In essence this acoustic technology can be regarded as a rapid screening tool to monitor the rinse behavior of compositional very different systems. It allows for rapid quantification of a qualitative attribute. Variations used is discussed below.

Finger-Tip Acoustic Experiment

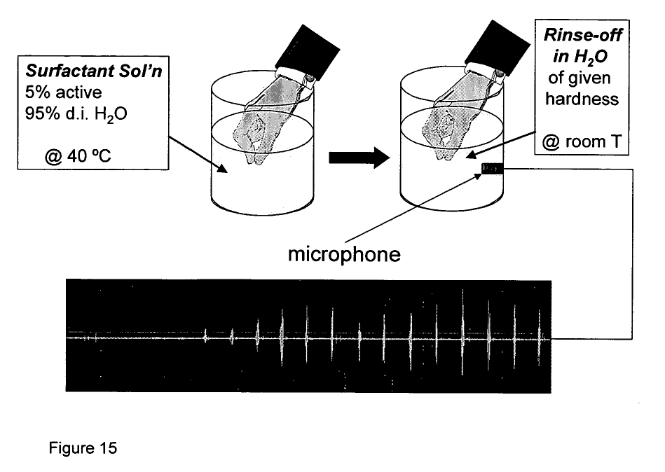

This method is used to monitor intensity of squeakiness of a given surfactant solution. FIG. 15 is an illustration of the procedure. The Bruel & Kjaer 8103 (Atlanta, GA) hydrophone (seen as an attachment on the container) receives an acoustic emission, as the fingertips are rubbed against each other in a given solution. This charged acoustic signal from the hydrophone is converted to a voltage signal via a Bruel & Kjae Conditioner Amplifier which is then converted to a digital signal to the computer via a Bruel & Kjaer Pulse 6.1 and 7.0 (Atlanta, Ga.) acoustic system. The intensity of the acoustic emission reflects the intensity of squeakiness for the solution.

Fore-Arm Wash Acoustic Experiment

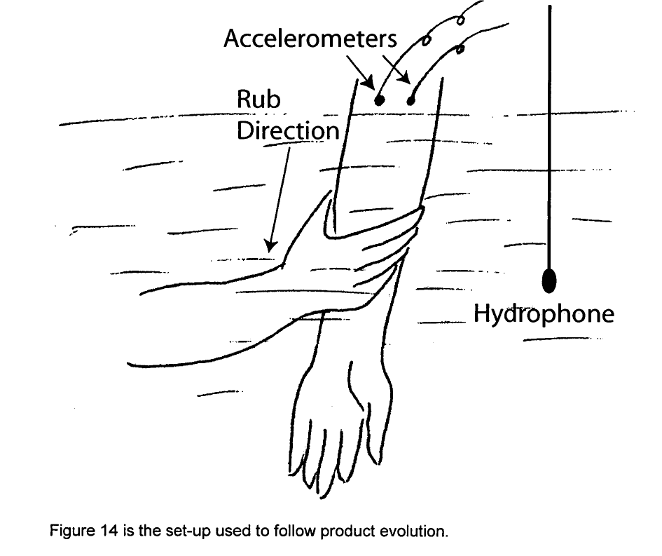

This method was used to monitor rinsability and “feel” of skin cleansers. FIG. 14 is an illustration of the typical set-up used in the assessment of wash-off (cleansing) products. Here, an acoustic signal is detected by a hydrophone as a product applied to the forearm is being rinsed off by the other hand. A typical procedure follows. A known amount of cleaner was applied on the wet forearm with the hand. The arm was then immersed into the rinse tank filled with water of a given hardness and temperature. Using the un-immersed hand the cleanser was then washed off by stroking downwards while the acoustic signal was recorded. Accelerometers (PCB 352A24) can also be attached to the subject's skin which allows one to follow vibrations during downward strokes (or rubbing) under water immersion. The charged acoustic signal from the hydrophone is converted to a voltage signal via a Bruel & Kjae Conditioner Amplifier which is then converted to a digital signal to the computer via a Bruel & Kjaer Pulse 6.1 and 7.0 (Atlanta, Ga.) acoustic system. The signal from the accelerometer were converted to a voltage signal via a PCB 442B104 amplifier (PCB Piezotronics, Inc., Depew, N.Y.).

Panel Study

Fourteen and twenty Japanese females (age range: 30-55 years old) voluntarily participated in the Three-point sensory study and Sensory study of calcium modified Dove bar respectively. The subjects were trained to recognize “squeaky-clean feel”. (“Kyu-Kyu” in Japanese consumer language. It is defined as resistance to moving the fingers on the skin).

Stimuli, Procedures and Questionnaires for the Three Point Sensory Study

TABLE 1
Three-Point Sensory Study

| | | Expected Order of |
|---|---|---|
| Solution | Composition | “Squeaky-Clean Feel” |
| A | 3.0% SDS + 0.12% CaCl₂ | The least |
| B | 1.5% SDS + 0.12% CaCl₂ | Medium |
| C | 0.3% SDS + 0.12% CaCl₂ | The most |

Subjects were asked to clean their hands with Kao White soap (composition as follows: 77.25% anhydrous 65/35 soap; 7.5% palm kernel oil fatty acid; 13.5% water, 4% fragrance; 0.75% whitener) before testing. After drying their hands the subjects dipped the thumb and index finger of one hand into a solution labeled A, B or C having compositions listed above in Table 1, while the thumb and index finger of the other hand was dipped into a separate solution also labeled A, B, or C. The subjects were then asked to rub the fingers of both hands in circular motion simultaneously and evaluate squeaky-clean feel between the solutions. After taking their fingers out of the solutions, the subjects answered a questionnaire designed specifically for this study. After a short rest interval of two minutes the subjects continued testing another pair of solutions.

Each subject compared and evaluated four sample pair (AB, AC, BC & BB). The presentation orders of the sample pairs were randomized and each solution was presented to the left and right arms equally across subjects. The data were analyzed using the Thrustonian approach to discrimination testing and categorical scaling. D-prime value (d′) was calculated and used to present sensory differences between samples. The bigger the d′, the more different the sample.

Stimuli, Procedures and Questionnaires for the Sensory Study of Calcium Modified Bar

| | | Expected Order of “Squeaky- |
|---|---|---|
| Bar | Composition | Clean Feel” Intensity |
| DEFI bar | See below* | The least |
| DEFI + Ca | Same + 4% CaCl₂ | Medium |
| Kao White | Kao White (as above) | The most |

Note:
All the bars were made by standard extrusion process.
*Formulation of DEFI base

| Ingredient | % |
|---|---|
| SCl | ≈50% |
| Free fatty acid | ≈23% |
| Fatty acid soap | ≈7% |
| Sodium isethionate | ≈5% |
| Sodium stearate | ≈3% |
| Betaine | ≈3% |
| Water & minors | Balance |

Subjects were asked to clean their hands and forearms with Kao White soap (control) at the beginning of the study. The subjects rinsed one arm under 60-ppm water thoroughly for 15 seconds; then the subjects washed the arm with the control bar and rinsed off according to the protocol used in quantitative descriptive analysis for wash-off product. The subjects were notified that the bar they washed which was the control bar. The subjects then washed the arm with a testing bar (DEFI or DEFI+Ca) labeled with a 3-digit random number. The subjects kept time using a digital stop watch from the beginning of washing until they perceived as squeaky-clean feel. When the subjects finished washing, they answered the questions in a questionnaire regarding squeaky-clean feel designed forth is study. The subjects washed the other arm using the other test bar and answered the other squeaky-clean question in the questionnaire.

After both arms were dried, the subjects were asked to compare and evaluate powdery feel on their washed forearms by rubbing their hands in an up-down motion. They answered questions regarding powdery feel of the forearms in the questionnaire.

In the last portion of the test protocol the subjects were asked to wash their hands under 60-ppm water and rub their hands on their dry forearms starting with a forearm that was washed first with a testing bar. The subjects compared and evaluated the sliminess of the forearms using the questionnaire.

Each subject compared and evaluated both samples two times in three separate days. Within each day, each samples evaluated equally (10 times). Across days and subjects, each bar was evaluated on right and left hands equally (30 times.).

Before any analysis, the data was checked for its quality by scrutinizing the first and the second questions (“Comparing the sample to the control, are the bars different in their squeaky-clean feel?” and “If yes, which bar gave you more squeaky-clean feel?”). Only subject who answer “Yes, they are different” and “Control was more squeaky-clean than the Dove sample” were subjected to further analyses. It is known that Kao White (a soap bar) elicits “squeaky-clean feel” in 60 ppm hard water while the samples (DEFI) elicits less squeaky-clean feel”. Therefore, subjects who thought that Kao White is less squeaky-clean than DEFI bar may use different criteria in judging the concept of squeaky-clean feel which is not the objective of this study and were thus eliminated from further study in this panel.

Time (seconds) and percent relative to Kao White data were analyzed using a repeated statistical model (ANOVA) with the bars as within subject effect. As before a Thurstonian model was used to analyze any 2-Alternative Choice with no difference optional type question (yes/no/no difference) and categorical rating was analyze during Thrustonian model aforementioned.

### Example 1

### Phase Diagram of SDS and Calcium Chloride (CaCl₂)

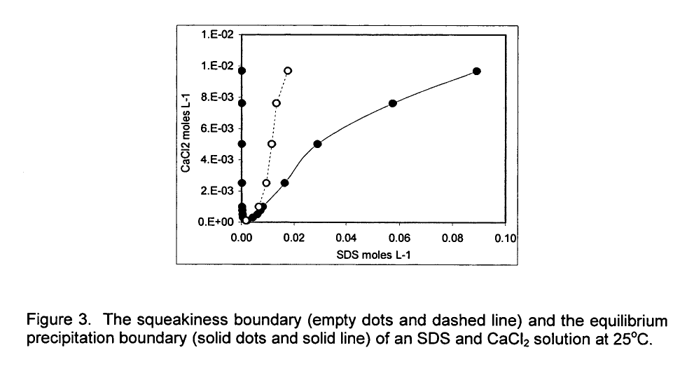

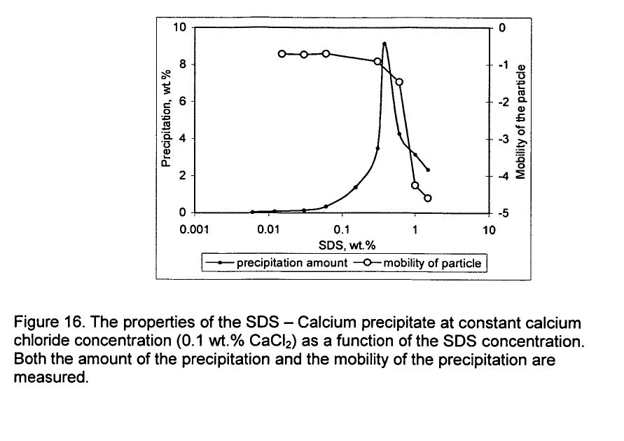

FIG. 3 shows the squeakiness boundary (empty dots and dashed line) and the equilibrium precipitation boundary (black dots and solid line) of an SDS and CaCl₂ solution at 25° C. FIG. 16 is the amount of the precipitation (▪) and the mobility of the precipitation (◯) of the SDS—Calcium precipitate at constant calcium chloride concentration (0.1 wt. % CaCl₂) as a function of the SDS concentration. FIG. 13, shows the change of surface tension within SDS-CaCl2 solution (at constant CaCl₂ concentration of 0.1 wt. %) and how the squeakiness boundary was defined. It was found that the surface tension remains at a characteristic value of micellar solution even after entering into the precipitation boundary. The surface tension then increases to a plateau region until it increases again to a characteristic value of pure water. A simple dye test also showed that micelles still exist right after entering into the precipitation region but micelles disappear with further dilution of the surfactant solution. Comparing the surface tension change (shown in FIG. 13) and the mobility of precipitate (shown in FIG. 16) for the same calcium concentration, it is found that, roughly at the same surfactant concentration, the surface tension increases and the charge of the surfactant—calcium precipitate decreases. This further indicates that the surface properties of the surfactant—calcium particles are closely related to the surface activity of the surfactant.

In general, the surface tension results shown in FIG. 13 suggest that, within the precipitation region, there is another characteristic region, namely squeakiness region. This squeakiness boundary identified by surface tension measurement, was plotted in FIG. 3, as noted, with the equilibrium precipitation boundary for SDS and CaCl₂ system. The squeakiness boundary, as noted, is a narrower region than the precipitation boundary. Thus, FIG. 3 “defines” a phase region.

### Example 2

### A Quantitative Measure of Squeakiness in SDS—CaCl₂ Phase Diagram: Finger-Tip Acoustic Test

As a quantitative measure of squeakiness, an Acoustic Test was done for different concentrations of SDS and Calcium Chloride falling in the four different regions as illustrated by the surfactant—Ca phase diagram (See FIG. 2).

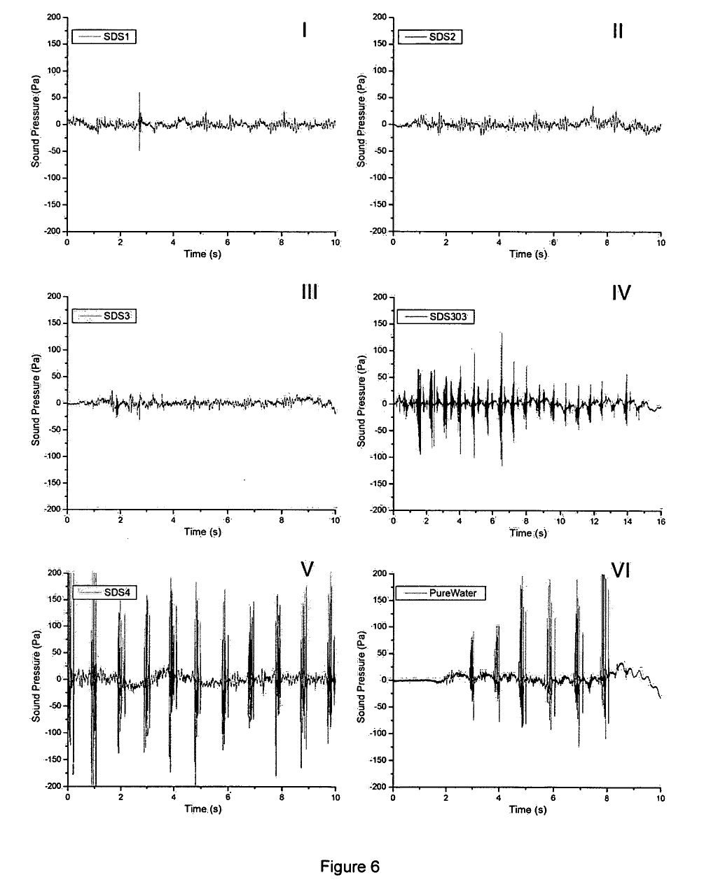

To illustrate the degree of squeakiness in these different regions as a function of surfactant concentration at a fixed calcium concentration, applicants conducted a simple fingertip experiments with sensory acoustics as described in the Protocol. FIGS. 5 and 6 show the results of SDS solution with and without Ca²⁺. FIG. 6 shows the acoustic profiles from the SDS/water system. In this case, no calcium salt was added. For all systems with the surfactant concentration above the CMC (FIG. 6I-III), the acoustic emissions are very low, which is indicative of slimy feel. As the concentration of the surfactant is below the CMC, the acoustic emission increases (FIG. 6IV). For the solution with a surfactant concentration well below the CMC, the acoustic pressure can reach values of 200 Pa (FIG. 6V-VI), indicating a squeaky feel.

For the SDS solution with calcium salt, the squeakiness profile along SDS concentration is totally different. FIG. 5 shows the acoustic profiles for the SDS/Ca-water system. A good correlation between FIG. 5 and FIG. 2(a) can be found. In Region A and B (FIG. 2(a)), sound pressure is very low corroborating the fact that the solutions in these two regions are very slimy. But for the solutions in Region C and D (FIG. 2(a)), sound pressure reaches a value of 150 Pa corroborating the fact that a very squeaky feel can be perceived.

Thus, it can be seen that surfactant calcium phase diagram predict regions of squeaky feel.

### Example 3

### A Quantitative Measure of Squeakiness in SDS—CaCl₂ Phase Diagram: Three-Point Sensory Test

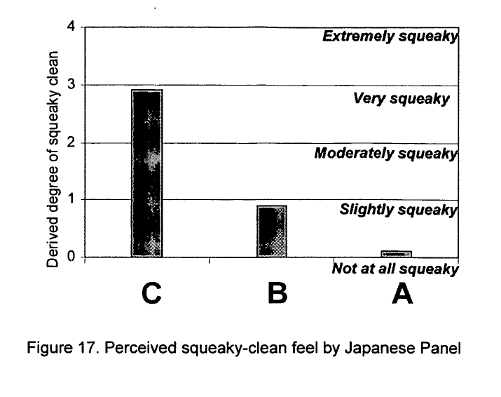

As a quantitative measure of squeakiness and to corroborate definition of different sensory regions in surfactant—salt phase diagram, a Three-point Sensory Study as described in the Protocol was done for samples falling in the three different regions A, B, and C as depicted in FIG. 2(a). The results are plotted in FIG. 17.

The results confirm that consumers can in fact perceive these differences in squeakiness as confirmed also by acoustic measurements. All solutions were significantly different from each other for squeaky-clean perception at a 95% confidence level. Using solution B as a reference point (d′=0.00), solution A was perceived as being significantly less squeaky-clean than solution B (d′=−1.58) while solution C was perceived as being significantly more squeaky-clean than solution B (d′=1.88). Therefore, solution C will be perceived as significantly more squeaky-clean than solution A (d′ of difference=1.88+1.58=3.46 which is very high value), FIG. 17.

TABLE 1
Example of formulation for the three point panel test

| Solution | Composition |
|---|---|
| A | 3.0% SDS + 0.12% CaCl₂ |
| B | 1.5% SDS + 0.12% CaCl₂ |
| C | 0.3% SDS + 0.12% CaCl₂ |

### Example 4

### Phase Diagram of SCI Type Surfactant and Calcium Chloride (CaCl₂)

The phase diagrams of two industrial grade SCI surfactants, Jordapon and DEFI, with SCI content around 85% and 72% (the rest is made up predominantly by fatty acid), were constructed. Because both Jordapon and DEFI have a Kraft temperature higher than the room temperature, their solutions at room temperature are cloudy already, which make it difficult to identify the precipitation boundary in the surfactant—calcium phase diagram by visual observation. Therefore, only the squeakiness boundary, identified by measuring the surface tension as stated before, was constructed in this study as shown in FIG. 4.

From FIG. 4, it was found that, compared to SDS, the squeakiness boundary of SCI types of surfactants covers a much smaller area in phase diagram (i.e., SCI is “slimy” compared to SDS). At a certain surfactant concentration, surface tension doesn't drop until a relatively high calcium salt concentration is reached. In other words, the SCI surfactants are less calcium sensitive compared to SDS surfactant. That probably is one of the reasons why SCI types of surfactants are perceived as slimy during rinsing under normal water hardness.

### Example 5

### Effect of Fatty Acid on the Squeakiness Region of Surfactant—Salt Phase Diagram: SCI Type of Surfactant—CaCl₂

As shown in FIG. 4 compared with Jordapon, DEFI (which has a higher fatty acid content) has the squeakiness region shifts to slightly higher surfactant concentration under certain calcium concentration. In other words, adding fatty acid to the SCI type of surfactant, the squeakiness region was enlarged as indicated by the phase diagram, which could potentially lead to a faster rinse (the squeakiness may happen earlier during rinsing). It is well known that SCI forms a solid complex structure with fatty acid, which might increase its calcium sensitivity to certain extent.

### Example 6

### Faster Rinsing by Preformulate Calcium Salt into Surfactant Containing Formulation

FIG. 2 (a) shows a schematic of how to achieve faster rinsing (squeakiness happens with less dilution) through the understanding of the surfactant—salt phase diagram. The Formulation 1 is a surfactant formulation with salt (calcium) preformulated. Formulation 2 is a surfactant formulation with little salt. As seen, upon dilution, Formulation 1 can achieve squeakiness as it goes through squeaky region (gray region C); while Formulation 2 will not be squeakiness until it reaches the CMC (critical micelle concentration) of the surfactant. In other words, much less dilution is needed for Formulation 1 to be perceived as squeaky compared to formulation 2.

### Example 7

### Faster Rinsing by Preformulating Calcium Salt into SDS Formulation

FIG. 3 shows the squeakiness region of SDS. From this phase diagram, similarly to the schematics shown in EXAMPLE 6, if calcium salt is preformulated into the SDS formulation, squeakiness may happen faster as the dilution route hits the squeakiness region defined by the phase diagram. For instance, comparing Formulation 1 (of FIG. 2) (0.5% SDS+0.1% CaCl2) to Formulation 2 (0.5% SDS solution, roughly two—time of dilution is need for Formulation 1 to achieve squeaky rinse (hits the squeakiness region in the phase diagram), while 25 times of dilution is needed for Formulation 2 to achieve squeaky rinse (hits the CMC of the surfactant).

### Example 8

### Faster Rinsing by Preformulating Calcium Salt into SCI Type of Surfactant

FIG. 4 shows the squeakiness region of SCI type of surfactant, Jordapon and DEFI, with SCI content around 85% and 72% (the rest is made up predominantly by fatty acid). From this phase diagram, similarly to the schematics shown in FIG. 2(a), if calcium salt is preformulated into the DEFI or Jordapon formulation, squeakiness may happen faster as the dilution route hits the squeakiness region defined by the phase diagram. It is difficult to ascribe an actual number here, as for Example 7, as DEFI or Jordapon exist in solution as crystal phase in equilibrium with the solution phase. In other words, the phase diagram is an equilibrium measurement, while in real use, kinetics become important).

### Example 9

### Faster Rinsing by Preformulating Calcium Salt into DEFI: A Fore-Arm Wash Acoustic Test

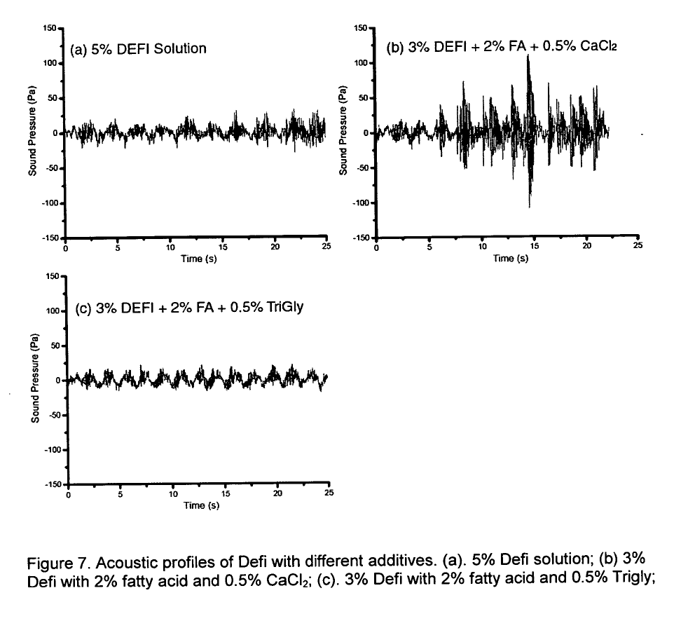

FIG. 7 is the acoustic profiles of DEFI alone (dilution route 2) vs. DEFI with fatty acid and calcium chloride preformulated (dilution route 1). It can be seen that for dilution route 1, less than 10 seconds are needed to achieve squeaky feel, as indicated by a stronger sound pressure; while for dilution route 2, squeaky feel is not achieved until the end of the experiment (25 seconds). The addition of fatty acid and Ca seems to improve the acoustic profile, FIG. 7. It begins to show an increase in the acoustic emission at the 4^{th} rub (>10 sec). Replacing calcium with an equivalent amount of triglyceride oil illustrates the importance of calcium in inducing precipitation of SCI leading to squeaky-clean perception, FIG. 7.

### Example 10

### Faster Rinsing by Preformulating Calcium Salt into DEFI: A Fore-Arm Wash Japanese Panel Test

A panel study was set up to compare the rinsing properties of a DEFI based bar modified with calcium chloride vs. a DEFI based bar (composition as defined for panel test above). For the Calcium Modified DEFI bar three perceptions were highlighted for study. These included “squeaky-clean feel”; “powdery feel”; and “slimy feel”. Also investigated in this study was the relationship between time of rinsing until a squeaky-clean feel was perceived i.e. time to rinse.

Average time to rinse of an arm washed with DEFI bar was 13.8 (11.9 to 15.6) seconds and average time to rinse of DEFI bar+4% CaCl₂ was 12.1 (10.3 to 13.8). Even though, DEFI+CaCl₂ was rinsed faster than DEFI bar, the differences were not significant (repeated measure F_{1,23}=2.04; p-value=0.17).*

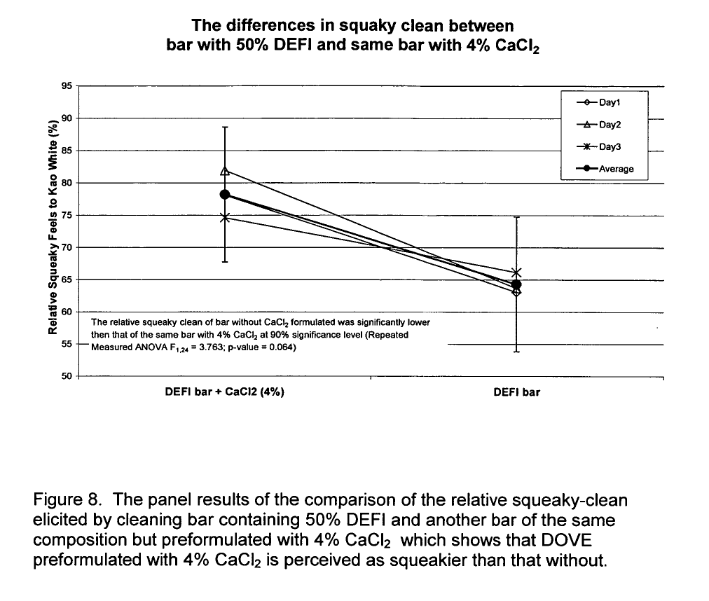

There was substantial evidence to support that the relative squeaky-clean feel of DEFI based bar+4% CaCl₂ was significantly higher than that of Dove (Repeated Measured ANOVA F_{1,24}=3.763; p-value=0.064), FIG. 8.

*F_{1,23} represents repeated measure F-ratio with numerator degree of freedom=1 and denominator degree of Freedom 23. Same for F_{1,24} below.

### Example 11

### Improve Surfactant Rinsing Properties by Blending Calcium Insensitive Surfactant with Calcium Sensitive Surfactant

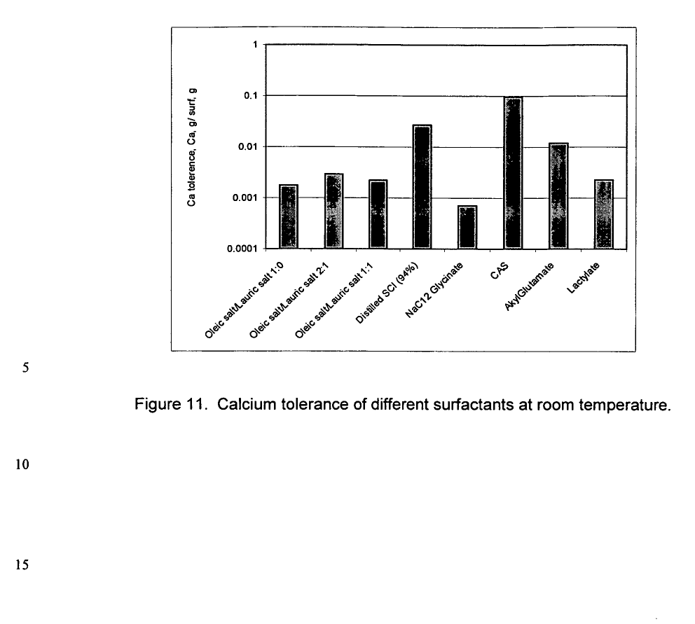

The calcium tolerance of various surfactants of interest was tested and the results are shown in FIG. 11. Soap surfactants (e.g., sodium laurate and sodium oleic) and their mixture, glycinate and lactylate have relatively low calcium tolerance (easier to form complex); SCI and CAS exhibit high calcium tolerance (hard to precipitate and form complex). It is believed low calcium tolerance is the predominant reason why soap surfactants and glycinate are typically perceived as squeaky. High calcium tolerance is one of the reasons why SCI is perceived as slimy and leaves slimy residue on skin after rinsing. CAS has very high calcium tolerance as shown in FIG. 11, but CAS is typically perceived as a very squeaky surfactant. The main reason probably is that CAS is a very water soluble surfactant (has low K.T.), that therefore the deposition of any surfactant structure on skin during wash is very unlikely to happen, since the surfactant activity reduces dramatically as the soluble surfactant is being quickly washed away during rinse. Again, it can be seen that there is a complex relationship between Krafft point and cation sensitivity and that the effect of one often counterbalances the effect of the other.

### Example 12

### Enhancing Squeaky Feel by Surfactant Chain Length

SCI (isethionate) surfactants of different chain length, ranging from C₁₀ to C₁₈, were examined for their rinsing properties from the point of view of calcium sensitivity and surfactant structure. Their Kraft temperatures (e.g. temperature at which surfactant crystals dissolve completely in solution) were also roughly estimated. The higher the Kraft Point (K.P.), the more crystals are found at room temperature. C₁₀ SCI has a Kraft temperature less than 20° C., and, therefore, no surfactant crystal structure is found at room temperature; C₁₂ SCI (distilled) has a Kraft temperature just around room temperature; C₁₄ around 45° C.; C₁₆ and C₁₈ both have a Kraft temperature higher than 55° C. The latter higher chain length isethionate have high crystal content at room temperature, which would normally be associated with deposition and enhanced “slimy” feeling. However, as discussed, cation sensitivity also plays a role on ultimate perception. Thus a C₁₆, C₁₈ chain length is highly cation sensitive, will form precipitate easily and be perceived as squeaky. Ideal chain length will be those either short (e.g., C₁₀) or long (e.g., >C₁₆). Those intermediate ones will be the most slimy, as neither structure nor calcium sensitivity act to its favor.

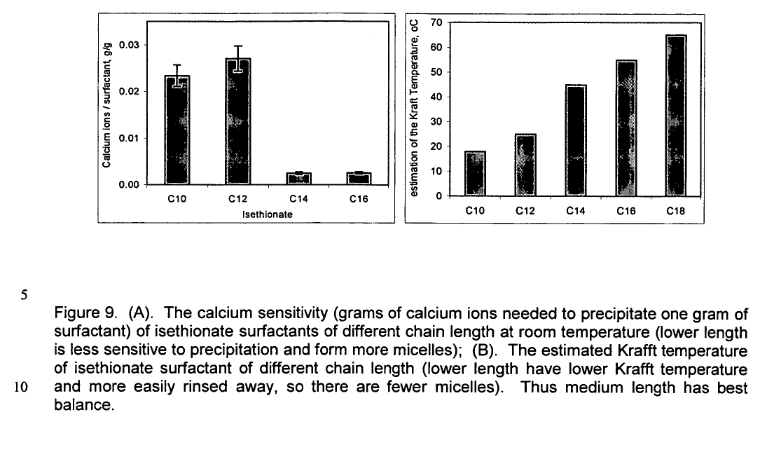

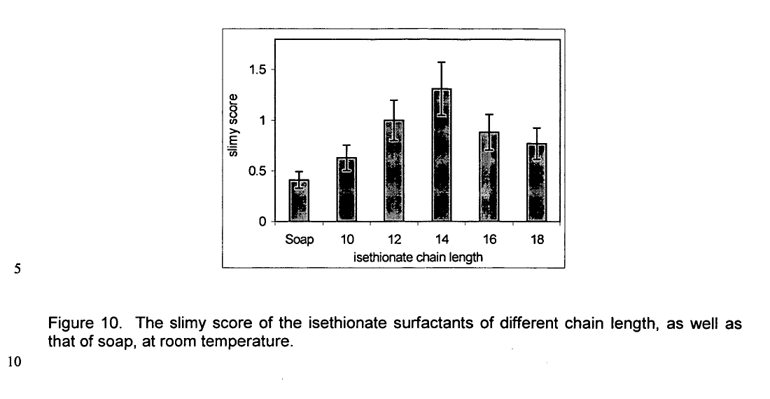

In FIG. 9, the calcium sensitivity data and the estimated Kraft temperatures of the SCI surfactants of various chain lengths are plotted. It was found that, with the increase of the chain length, the calcium sensitivity increases (easier to precipitate complex and be squeaky). However, the surfactant structure at room temperature also increases (more deposition and thus more “slimy”) as the Kraft temperature becomes higher as just discussed above. In FIG. 10, the slimy score of the SCI surfactants with different chain length is reported. The slimy score is low (the formulation was perceived as squeakier than others) at low chain length, when there is no surfactant crystal structure present in the system even though the calcium sensitivity is relatively low (e.g., even the lack of sensitive/precipitation is normally associated with sliminess), since there is little or no crystal structure, and there is nothing to deposit and cause sliminess; the slimy score is also low at higher chain length end, when the calcium sensitivity is very high, even though plenty of surfactant crystal structure is present in the formulation (precipitate-complex formation and resulting “squeakiness” swamps at the effect of more surfactant micelles being around to deposit and enhance slime). The formulation was perceived as most slimy at intermediate surfactant chain length, where neither cation sensitivity nor surfactant crystal structure becomes dominantly favorable to the squeakiness.

Based on the above learning, two technical approaches can be proposed in order to improve the squeaky feel of a surfactant solution: 1). Promote the surfactant—cation interaction either by increasing the cation sensitivity of the surfactant (such as by increasing the surfactant hydrophobic chain length from, for example, C₁₂ to C₁₆ or C₁₈) or by preformulating cation salt into the surfactant formulation; and/or 2) by breaking the surfactant solid structure to reduce the deposition of the surfactant solid onto skin by methods such as using surfactant blending, cosolvent or small molecular additives; or using smaller chain length surfactant with lower KT.

### Example 13

### Enhancing Squeaky Feel by Addition of Fatty Acid

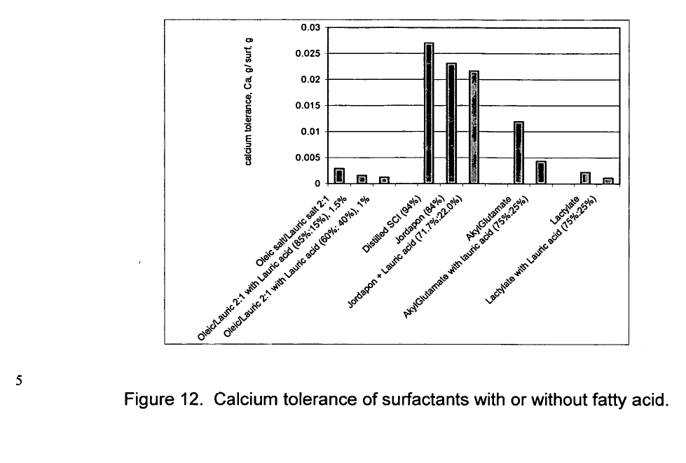

In FIG. 12, the calcium tolerance of surfactant alone and that of surfactant with fatty acid are compared at room temperature. As shown in FIG. 4, adding fatty acid into a surfactant formulation tends to reduce the calcium tolerance of that surfactant to some extent (more easy to form complex) and probably will lead to squeaky feel during wash. However, the Kraft temperature of most surfactant/fatty acid complex will be higher than surfactant alone (more crystals around to deposit). So adding fatty acid may also lead to more structure in the formulation and promote the deposition, which cause slimy feel during rinsing. Again, the complexity of the relationship can be seen.

1. Cleanser composition containing at least one anionic surfactant and at least one multivalent cation containing salt such that the composition during rinsing passes through a region of a phase diagram where precipitation of surfactant-multivalent salt occurs whereby the solution is depleted of micelles at fewer dilutions than required to achieve a micelle-free solution in the absence of the multivalent cation containing salt.

2. A composition according to claim 1, wherein said surfactant-cation precipitate which leads to fewer dilutions to achieve depletion of micelle is formed by increasing the interaction/precipitation between said surfactant and available salt counter-ion.

3. A composition according to claim 2, wherein said interaction is increased by increasing level of available counter-ion in a solution comprising said surfactant.

4. A composition according to claim 3, wherein the increased counter-ion is preformulated into the surfactant solution.

5. A composition according to claim 2, wherein said interaction is increased by increasing the sensitivity of said surfactant to precipitation by the counter-ion.

6. A composition according to claim 5, wherein said sensitivity is increased by using hydrophobic group of chain length C₁₆ or greater.

7. A composition according to claim 2, wherein said counter-ion is a cation.

8. A composition according to claim 7, wherein said cation is calcium.

9. A composition according to claim 7, wherein the cation is aluminum.

10. A composition according to claim 1, resulting enhanced squeaky feel sensation.
